(19) United States
(12) Patent Application Publication
(10) Pub. No.: US 2018/0221128 A1
(43) Pub. Date: Aug. 9, 2018

(54) SYSTEM, METHOD, AND APPARATUS FOR COLLECTING EMBOLI

(71) Applicant: THE CLEVELAND CLINIC FOUNDATION, Cleveland, OH (US)

(72) Inventors: Mehdi H. Shishehbor, Pepper Pike, OH (US); Marwane Berrada-Sounni, Beachwood, OH (US); Bruce L. Wilkoff, Chagrin Falls, OH (US)

(21) Appl. No.: 15/890,489

(22) Filed: Feb. 7, 2018

Related U.S. Application Data
(60) Provisional application No. 62/455,672, filed on Feb. 7, 2017.

(51) Int. Cl. A61F 2/01

(52) U.S. Cl. CPC A61F 2/01; A61F 2002/011; A61F 2002/016; A61F 2250/0063; A61F 2210/0014; A61F 2220/0008; A61F 2220/0025; A61F 2002/018

(57) ABSTRACT

Systems, methods, and apparatuses for collecting emboli include an embolic dual-filtration device are disclosed. The embolic dual-filtration device has a first filter and a second filter. The first and second filters have pores. The second filter is positioned adjacent to the first filter. The first and second filters are capable of being selectively rotated with respect to one another. The first and second filter pores of the rotated first and second filters collectively form a moiré lattice structure. The moiré lattice structure has pores smaller than the pores of each of the separate first and second filters.

### RELATED APPLICATION

This application claims priority from U.S. Provisional Application No. 62/455,672, filed 7 Feb. 2017, the subject matter of which is incorporated herein by reference in its entirety.

### TECHNICAL FIELD

This disclosure relates to a system, apparatus, and method for collecting emboli and, more particularly, to an embolic dual-filtration device and method for use.

### BACKGROUND

During procedures such as, but not limited to, thrombectomy, atherectomy, balloon angioplasty, stent deployment, and/or cardiac lead extraction, debris such as plaque, blood clots, vegetation, and debris associated with cardiac lead extraction can move from the treatment site through a vein or artery and compromise the flow of blood at a location downstream from the treatment site by creating an embolism. Various embolism protection systems may be used to help prevent such debris from traveling and/or embolizing within a vessel such as filters and occlusive devices.

Currently, embolic protection systems are commonly used for coronary, carotid, and peripheral procedures. The application of currently existing embolic protection systems to protect a pulmonary artery may be undesirable due to unsuitable designs and the incompatibility of the filters of the existing embolic protection systems to accomplish the desired filtration function in the pulmonary artery.

### SUMMARY

In an aspect, an embolic dual-filtration device is provided. The embolic dual-filtration device has a first filter and a second filter. Each of the first and second filters has pores. The second filter is positioned adjacent to the first filter. The first and second filters are capable of being selectively rotated with respect to one another. The first and second filter pores of the rotated first and second filters collectively form a moiré lattice structure. The moiré lattice structure has pores smaller than the pores of each of the separate first and second filters.

In an aspect, a system for collecting emboli in the pulmonary artery is provided. The system has a first filter. The first filter has a first filter support structure and a first filter mesh. The first filter support structure is capable of engaging patient pulmonary artery tissue. The first filter mesh has pores and is attached to at least a portion of the first filter support structure. The system has a second filter. The second filter has a second filter support structure and a second filter mesh. The second filter support structure is capable of engaging patient pulmonary artery tissue. The second filter mesh has pores and is attached to at least a portion of the second filter support structure. The second filter is positioned longitudinally adjacent to the first filter. The first and second filters are coaxially arranged relative to one another. The first and second filters are capable of being rotated with respect to one another once positioned in the patient pulmonary artery. The system includes a catheter configured to access the patient pulmonary artery. The catheter has a catheter lumen. The catheter lumen is configured to allow the first and second filters to pass therethrough. The first and second filter meshes of the rotated first and second filters collectively form a moiré lattice structure. The moiré lattice structure has pores smaller than the pores of each of the separate first and second filters.

In an aspect, a method for collecting emboli is provided. An embolic dual-filtration device is provided. The embolic dual-filtration device has a first filter and a second filter. Each of the first and second filters has pores. The second filter is positioned adjacent to the first filter. The first and second filters are capable of being selectively rotated with respect to one another. The embolic dual-filtration device is inserted into a patient pulmonary artery. The embolic dual-filtration device is maintained in the patient pulmonary artery. With the embolic dual-filtration device maintained in the patient pulmonary artery, the first and second filters are independently, selectively rotated to collectively form a moiré lattice structure. The moiré lattice structure has varying sized pores relative to the independent rotation of the first and second filters. The force of blood flow within the patient pulmonary artery is utilized to restrict blood-carried emboli that are larger than the pores of the moiré lattice structure to a location on an upstream side of the moiré lattice structure. The first filter, the second filter, and the restricted emboli are removed from the patient pulmonary artery. The emboli restricted to the upstream side of the moiré lattice structure are removed from the patient pulmonary artery when the first and second filters are removed from the patient pulmonary artery.

### BRIEF DESCRIPTION OF THE DRAWINGS

For a better understanding, reference may be made to the accompanying drawings, in which:

FIG. 1 is a side view of a embolic dual-filtration device according to one aspect of the present invention;

FIG. 2 is a top view of an element of the aspect of FIG. 1;

FIG. 3 is a top view of an element of the aspect of FIG. 1;

FIG. 4 is a side view of the elements of FIGS. 2-3 in a first use configuration;

FIG. 5 is a top view of the elements of FIGS. 2-3 in a second use configuration;

FIGS. 6-7 illustrate an example sequence of operation of a portion of the aspect of FIG. 1;

FIG. 8 is a partial cross sectional view of an element of the aspect of FIG. 1;

FIGS. 9-10 illustrate an example sequence of operation of a portion of the aspect of FIG. 1;

FIG. 11 is a side view of an element of the aspect of FIGS. 9-10 in a first use configuration;

FIG. 12 is a side view of an element of the aspect of FIG. 1 in a first use configuration;

FIG. 13-17 depict an example sequence of operation of the aspect of FIG. 1; and

FIG. 18 illustrates an example operation feature of the aspect of FIGS. 9-11.

### DESCRIPTION OF EMBODIMENTS

Unless defined otherwise, all technical and scientific terms used herein have the same meaning as is commonly understood by one of skill in the art to which the present disclosure pertains.

As used herein, the term “patient” can refer to any warm-blooded organism including, but not limited to, human beings, pigs, rats, mice, dogs, goats, sheep, horses, monkeys, apes, rabbits, cattle, farm animals, livestock, birds, etc.

As used herein, the term “user” can be used interchangeably to refer to an individual who prepares for, assists, and/or performs a procedure.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” can be interpreted to include X and Y.

It will be understood that when an element is referred to as being “on,” “attached” to, etc., another element, it can be directly on or attached to the other element or intervening elements may also be present. It will also be appreciated by those of skill in the art that references to a structure or feature that is disposed “adjacent” another feature may not have portions that overlap or underlie the adjacent feature. Further, it will be understood that when an element is referred to as being “adjacent” to another element, it can be contacting or spaced apart from the other element.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or Figures unless specifically indicated otherwise.

The invention comprises, consists of, or consists essentially of the following features, in any combination.

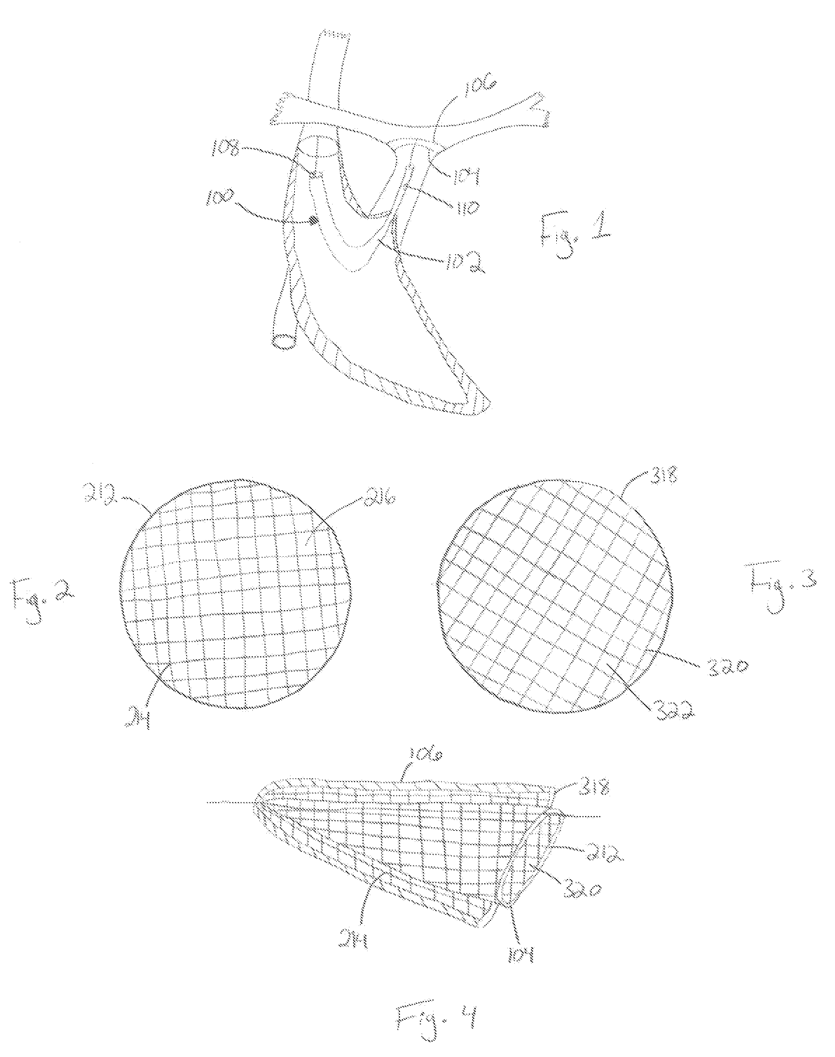

FIG. 1 depicts an embolic dual-filtration device 100. The embolic dual-filtration device 100 includes a catheter 102, a first filter 104, and a second filter 106. The catheter 102 is configured to access a patient pulmonary artery P. The catheter 102 has a catheter lumen 108 configured to allow the first and second filters 102, 104 to pass therethrough. The catheter 102 may have a side window 110 for allowing dye from the catheter lumen 108 to be injected into the patient pulmonary artery P.

As shown in FIG. 2, the first filter 104 has a first filter support structure 212 and a first filter mesh 214. The first filter mesh 214 may be attached to at least a portion of the first filter support structure 212. Instead of, or in addition to, a separate support structure, the first filter support structure 212 may be at least partially formed from the first filter mesh 214. The first filter support structure 212 is capable of engaging patient pulmonary artery tissue T, such as in, but not limited to, a press-fit engagement. The first filter support structure 212 is capable of conforming to the geometry of the patient pulmonary artery tissue T. The first filter mesh 214 has pores 216. The first support structure 212 formed at least partially from the first filter mesh 214 may have pores 216. The size of the pores 216 in the first filter mesh 214 may be at least 250 microns, and may be larger than 250 microns for many use environments.

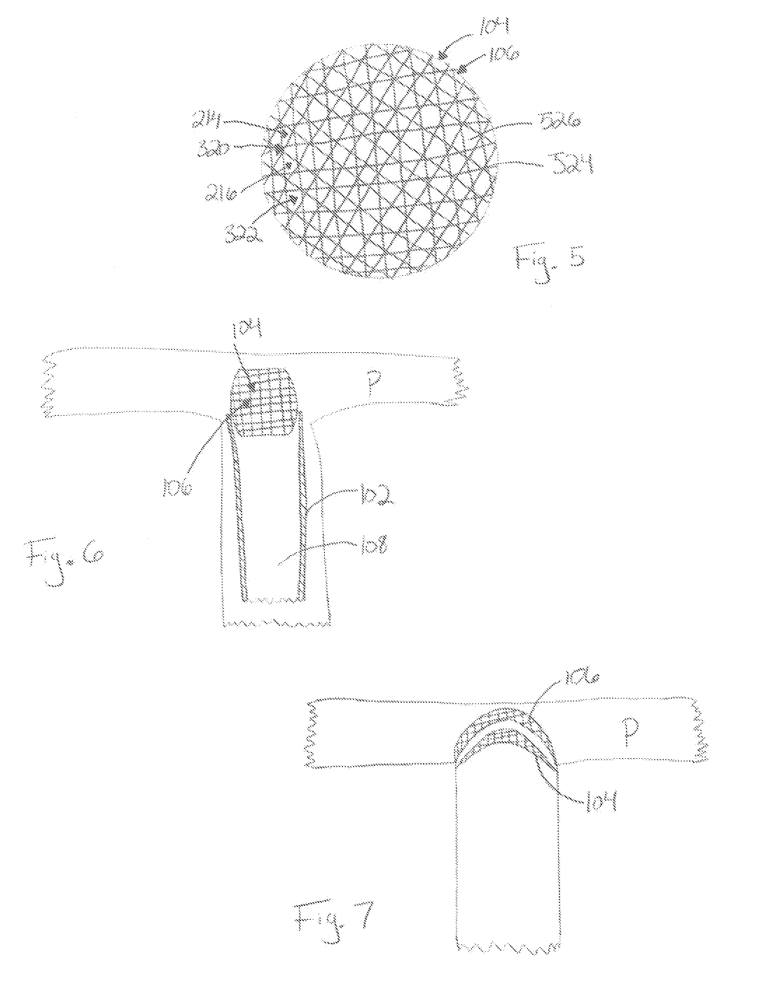

As shown in FIG. 3, the second filter 106 has a second filter support structure 318 and a second filter mesh 320. The second filter mesh 320 may be attached to at least a portion of the second filter support structure 318. Instead of, or in addition to, a separate support structure, the second filter support structure 318 may be at least partially formed from the second filter mesh 320. The second filter support structure 318 is capable of engaging patient pulmonary artery tissue T, such as, but not limited to, via a press-fit engagement. The second filter support structure 318 is capable of conforming to the geometry of at least one of the patient pulmonary artery tissue T and the first filter 104.

The second filter mesh 320 has pores 322. The second support structure 318 formed at least partially from the second filter mesh 320 may have pores 322. The size of the pores 322 in the second filter mesh 320 may be larger than 250 microns. The size of the pores 322 in the second filter mesh 320 may be the same size as the pores 216 in the first filter mesh 214 or may be smaller than the pores 216 in the first filter mesh 214. The second filter 106 may be positioned longitudinally adjacent to the first filter 104. The term “longitudinal” is used herein to indicate a substantially vertical direction, in the orientation of FIG. 1.

The first and second filters 104, 106 may be coaxially arranged relative to one another. The term “coaxially arranged” is used herein to indicate a positioning in which two or more elements have the same radical axis and/or centroid, such as the positioning of the first and second filters 104, 106 as shown in FIG. 1. The first and second filters 104, 106 may be disk shaped, cone shaped (as shown in FIG. 4), or any other suitable shape for engaging patient pulmonary artery tissue T and collecting emboli. The first and second filters 104, 106 are capable of being selectively rotated with respect to one another once positioned in the patient pulmonary artery P. At least one of the first and second filters 104, 106 may be selectively rotated with respect to the other of the first and second filters 104, 106 in any desired manner, with or without an interaction by the user, such as by a mechanism that is triggered to automatically rotate at least one of the first and second filters 104, 106, and/or by the user manually rotating at least one of the first and second filters 104, 106 directly, indirectly, or both.

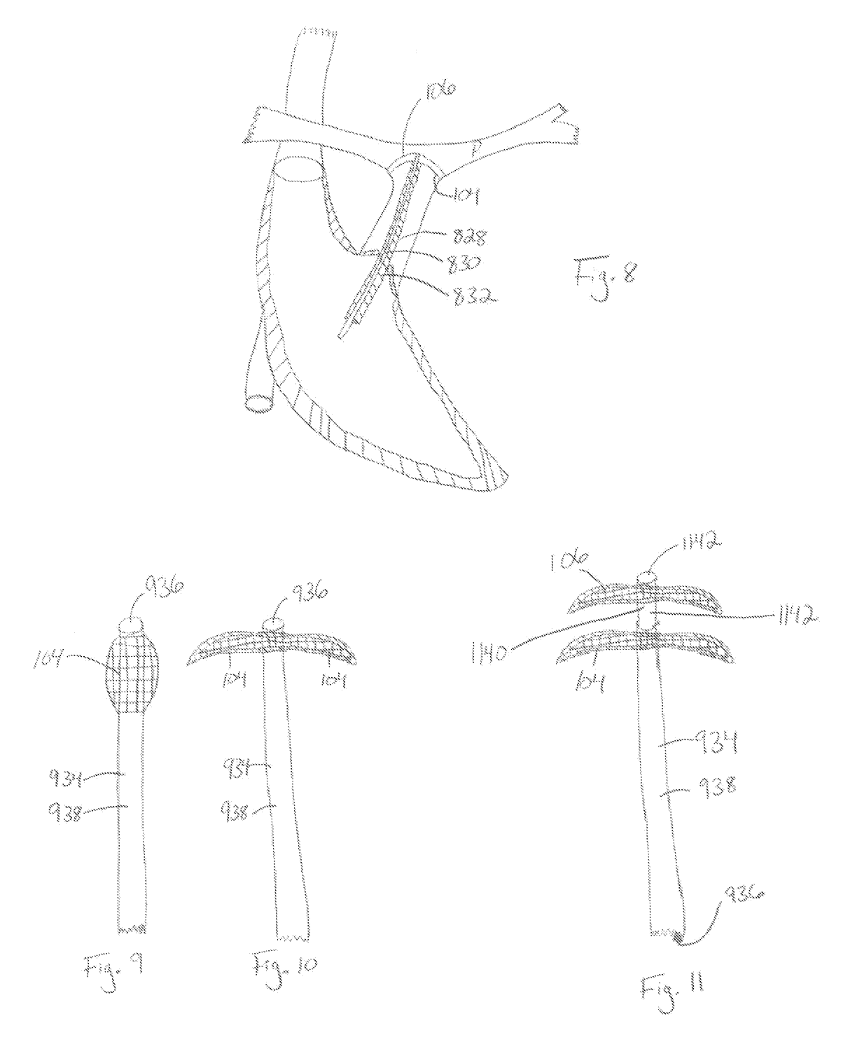

As shown in FIG. 5, the first and second filter meshes 214, 320 of the rotated first and second filters 104, 106 collectively form a moiré lattice structure 524 having pores 526 smaller than the pores 216, 322 of each of the separate first and second filters 104, 106. The term “moiré” is defined herein as the effect produced when two or more repetitive patterns of lines, circles, or array of dots are overlapped with imperfect alignment as shown in, for example, FIG. 5. At least one of the first and second filters 104, 106 may be selectively rotated, as previously described, to selectively adjust at least one of the size and shape of the pores 526 of the moiré lattice structure 524.

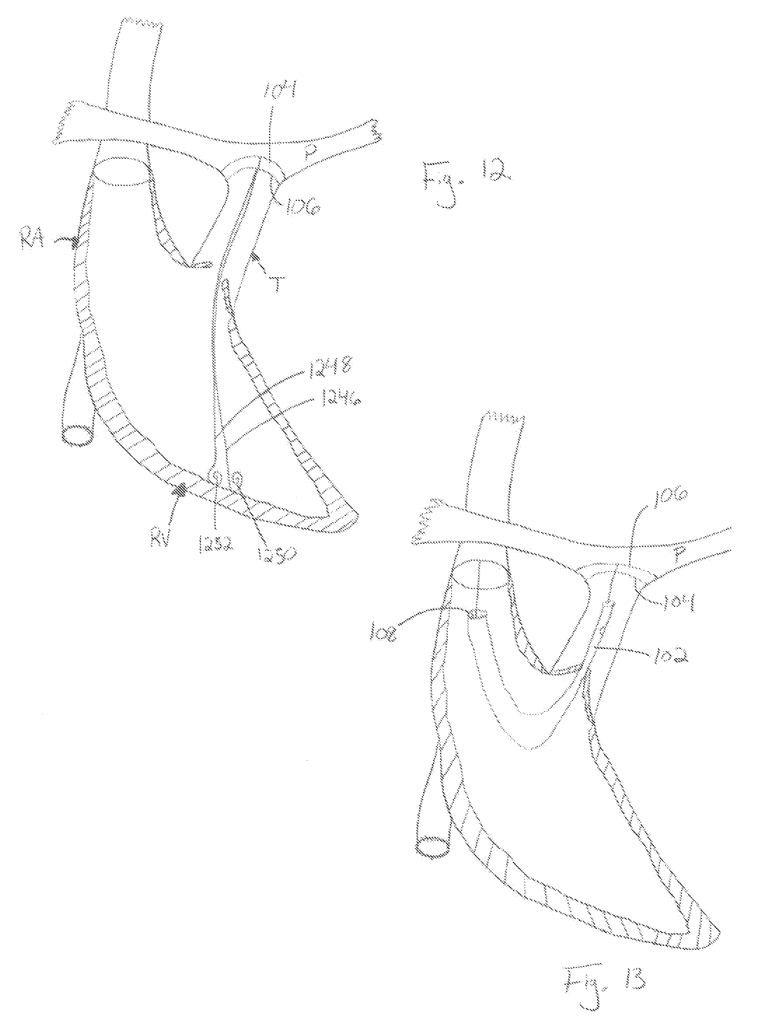

The first and second filters 104, 106 may each be formed at least partially from a deformable material. The deformable material may be elastic and/or a shape memory alloy, such as, but not limited to, nitinol. As shown in FIGS. 6-7, the first and second filters 104, 106 are each capable of being selectively moved between collapsed and expanded conditions. As shown in FIG. 6, in the collapsed condition, the first and second filters 104, 106 each define a first size profile that is configured for passage through the catheter lumen 108 to a desired location within a patient pulmonary artery P. As shown in FIG. 7, in the expanded condition, the first and second filters 104, 106 each define a second size profile that is configured to engage patient pulmonary artery tissue T. The first size profile of the collapsed condition is laterally smaller in width than the second size profile of the expanded condition. The term “lateral” is used herein to indicate a direction substantially perpendicular to the “longitudinal” direction, and is shown as the horizontal direction in the orientation of FIGS. 6-7.

The embolic dual-filtration device 100 may, for example, be collapsed into the collapsed condition by cooling the first and second filters 104, 106 to a temperature below a transition temperature range of the shape memory alloy. The first and second filters 104, 106 may be formed into the expanded condition as a first predetermined shape above a transition temperature range, the transition temperature range being dependent on the particular ratio of metals in the alloy. Below the transition temperature range, the alloy is highly ductile and may be plastically deformed into a second desired shape, such as the collapsed condition. Upon reheating above the transition temperature range, the alloy returns to its first predetermined shape, such as the expanded condition.

The embolic dual-filtration device 100 may also or instead be collapsed into the collapsed condition by the user and/or catheter lumen providing a laterally and/or longitudinally inward force on each of the first and second filters 104, 106. The dimensions of the catheter lumen 108, which are laterally smaller than the expanded embolic dual-filtration device 100, prevent the embolic dual-filtration device 100 from moving to the expanded condition when the embolic dual-filtration device 100 is passed through the catheter lumen 108. The embolic dual-filtration device 100 returns to its expanded condition when the laterally and/or longitudinally inward force provided by the user and/or the catheter lumen is removed. Further, the embolic dual-filtration device 100 may be collapsed/expanded to its collapsed/expanded condition in any desired manner, with or without an interaction by the user, such as by a mechanism that is triggered to automatically collapse/expand the embolic dual-filtration device 100, and/or by the user manually collapsing/expanding the embolic dual-filtration device 100 directly, indirectly, or both.

As shown in FIG. 8, the embolic dual-filtration device 100 may include a first drive shaft 828 and a second drive shaft 830. The first drive shaft 828 is attached to the first filter 104. The first drive shaft 828 has a first drive shaft lumen 832. The second drive shaft 830 is attached to the second filter 106. The second drive shaft 830 may be configured to fit within the first drive shaft lumen 832. The selective rotation of the first drive shaft 828 causes the first filter 104 to responsively rotate. The selective rotation of the second drive shaft 830 causes the second filter 106 to responsively rotate with respect to the first filter 104. At least one of the first and second drive shafts 828, 830 may be selectively rotated in any desired manner, with or without an interaction by the user, such as by a mechanism that is triggered to automatically rotate at least one of the first and second drive shafts 828, 830, and/or by the user manually rotating at least one of the first and second drive shafts 828, 830 directly, indirectly, or both.

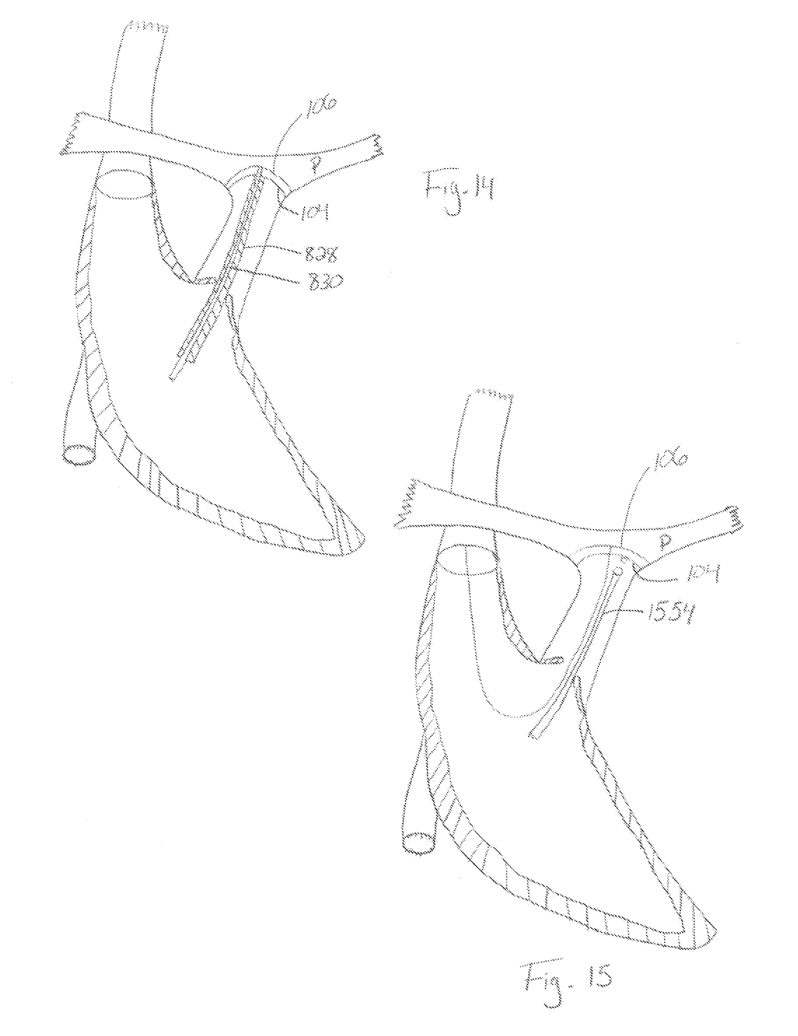

As shown in FIG. 9, the embolic dual-filtration device 100 may include a first filter deployment tool 934. The first filter deployment tool 934 has a first filter deployment tool inner lumen 936 and a first filter deployment tool outer wall 938. The first filter deployment tool outer wall 938 has the first filter 104 attached thereon. As shown in FIGS. 9-10, the first filter 104 is capable of being selectively moved between collapsed and expanded conditions around the first filter deployment tool outer wall 938 in a similar manner to that previously described. The first filter deployment tool 934 and attached first filter 104 may be configured to pass through the catheter lumen 108 when the first filter 104 is in the collapsed position. The first filter deployment tool 934 may include a selectively attachable and/or removable first filter deployment tool holding structure (not shown) that may be attached to at least one of the first filter deployment tool 934 and the first filter 104 to restrict the first filter deployment tool 934 to the collapsed condition. The first filter deployment tool holding structure may be selectively attached/removed in any desired manner, with or without an interaction by the user, such as by a mechanism that is triggered to automatically attach/remove the first filter deployment tool holding structure, and/or by the user manually attaching/removing the first filter deployment tool holding structure directly, indirectly, or both.

As shown in FIG. 11, the embolic dual-filtration device 100 may include a second filter deployment tool 1140. The second filter deployment tool 1140 has a second filter deployment tool inner lumen 1142 and a second filter deployment tool outer wall 1144. The second filter deployment tool outer wall 1144 has the second filter 106 attached thereon. The second filter 106 is capable of being selectively moved between collapsed and expanded conditions around the second filter deployment tool outer wall 1144 in a similar manner to that described for the first filter deployment tool 934. The second filter deployment tool 1140 may include a selectively attachable and/or removable second filter deployment tool holding structure (not shown) that may be attached to at least one of the second filter deployment tool 1140 and the second filter 106 to restrict the second filter deployment tool 1140 to the collapsed condition. The second filter deployment tool holding structure may be selectively attached/removed in any desired manner, with or without an interaction by the user, such as by a mechanism that is triggered to automatically attach/remove the second filter deployment tool holding structure, and/or by the user manually attaching/removing the second filter deployment tool holding structure directly, indirectly, or both.

The second filter deployment tool 1140 and attached second filter 106 may be configured to pass through at least one of the first filter deployment tool inner lumen 936 and the catheter lumen 108 when the second filter 106 is in the collapsed position. As shown in FIG. 11, once the second filter is passed through the first filter deployment tool inner lumen 936, the second filter 106 may be expanded into the expanded condition.

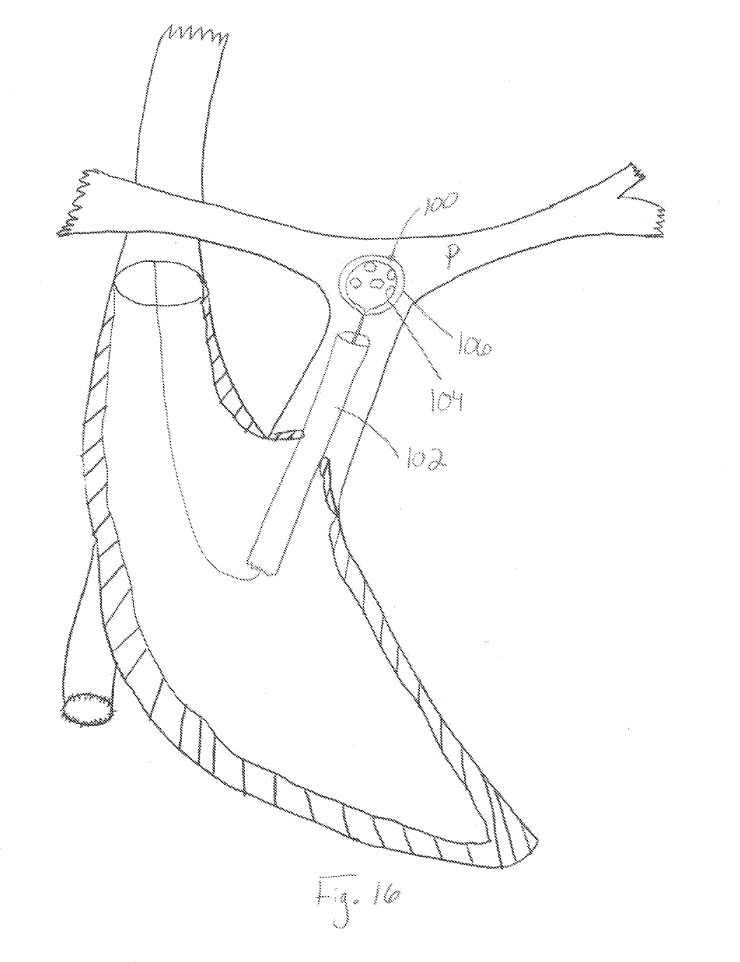

As shown in FIG. 12, the embolic dual-filtration device 100 may include a first anchoring member 1246 and a second anchoring member 1248. The first anchoring member 1246 is attached to the first filter 104. The first anchoring member 1246 has a first tissue engagement member 1250. The first tissue engagement member 1250 of the first anchoring member 1246 is capable of selectively anchoring the first filter 104 to at least one of patient pulmonary artery tissue T, patient right ventricle tissue RV, and patient right atrium tissue RA. The second anchoring member 1248 is attached to the second filter 106. The second anchoring member 1248 has a second tissue engagement member 1252. The second tissue engagement member 1252 of the second anchoring member 1248 is capable of selectively anchoring the second filter 106 to at least one of patient pulmonary artery tissue T, patient right ventricle tissue RV, and patient right atrium tissue RA.

In use, the embolic dual-filtration device 100, as described above, is provided to the user. The embolic dual-filtration device 100 is collapsed into the collapsed condition. The embolic dual-filtration device 100 may be collapsed into the collapsed condition by cooling of the first and second filters 104, 106 to a temperature below a transition temperature range of a shape memory alloy. The embolic dual-filtration device 100 may also or instead be collapsed into the collapsed condition by provision of a laterally and/or longitudinally inward force on each of the first and second filters 104, 106. The embolic dual-filtration device 100 may also or instead be collapsed into the collapsed condition in any desired manner, with or without an interaction by the user, such as by a mechanism that is triggered to automatically collapse the embolic dual-filtration device 100, and/or by the user manually collapsing the embolic dual-filtration device 100 directly, indirectly, or both. As shown in FIG. 13, the catheter 102 may be inserted into the patient pulmonary artery P. The catheter 102 may be inserted along a guidewire that was previously inserted into the patient pulmonary artery P.

With the embolic dual-filtration device 100 in the collapsed condition, the embolic dual-filtration device 100 is passed through the catheter lumen 108 and inserted into the patient pulmonary artery P. The embolic dual-filtration device 100 may be passed along the previously inserted guidewire. The embolic dual-filtration device 100 may be restricted from expanding while being passed through the catheter lumen 108 by the dimensions of the catheter lumen 108 being too small to allow the embolic dual-filtration device 100 to be moved into the expanded condition.

With the embolic dual-filtration device 100 in the patient pulmonary artery P, the embolic dual-filtration device 100 is expanded into the expanded condition in the patient pulmonary artery P. The embolic dual-filtration device 100 may be expanded by exposure of the embolic dual-filtration device 100 to blood having a temperature greater than the transition temperature range of the shape memory alloy. The embolic dual-filtration device 100 may be expanded by allowing the embolic dual-filtration device 100 to self-expand once unrestricted by the catheter lumen 108. The embolic dual-filtration device 100 may also be expanded in any desired manner, with or without an interaction by the user that causes the embolic dual-filtration device 100 to expand once properly positioned in the patient pulmonary artery P, such as by a mechanism that is triggered to automatically expand the embolic dual-filtration device 100, and/or by the user manually expanding the embolic dual-filtration device 100 directly, indirectly, or both.

The embolic dual-filtration device 100 is maintained in the patient pulmonary artery P. The embolic dual-filtration device 100 may be maintained in the patient pulmonary artery P by selective attachment of the first and second tissue engagement members 1250, 1252 to at least one of patient pulmonary artery tissue T, patient right ventricle tissue RV, and patient right atrium tissue RA. When the first and second tissue engagement members 1250, 1252 are attached to at least one of patient pulmonary artery tissue T, patient right ventricle tissue RV, and patient right atrium tissue RA, the first and second anchoring members 1246, 1248 hold the embolic dual-filtration device 100 in the patient pulmonary artery P to prevent the embolic dual-filtration device 100 from egressing from a desired position.

With the embolic dual-filtration device 100 being maintained in the patient pulmonary artery P, the first and second filters 104, 106 are independently and selectively rotated, in a similar manner to that previously described, to collectively form a moiré lattice structure 524 having varying sized pores 526 relative to the independent rotation of the first and second filters 104, 106.

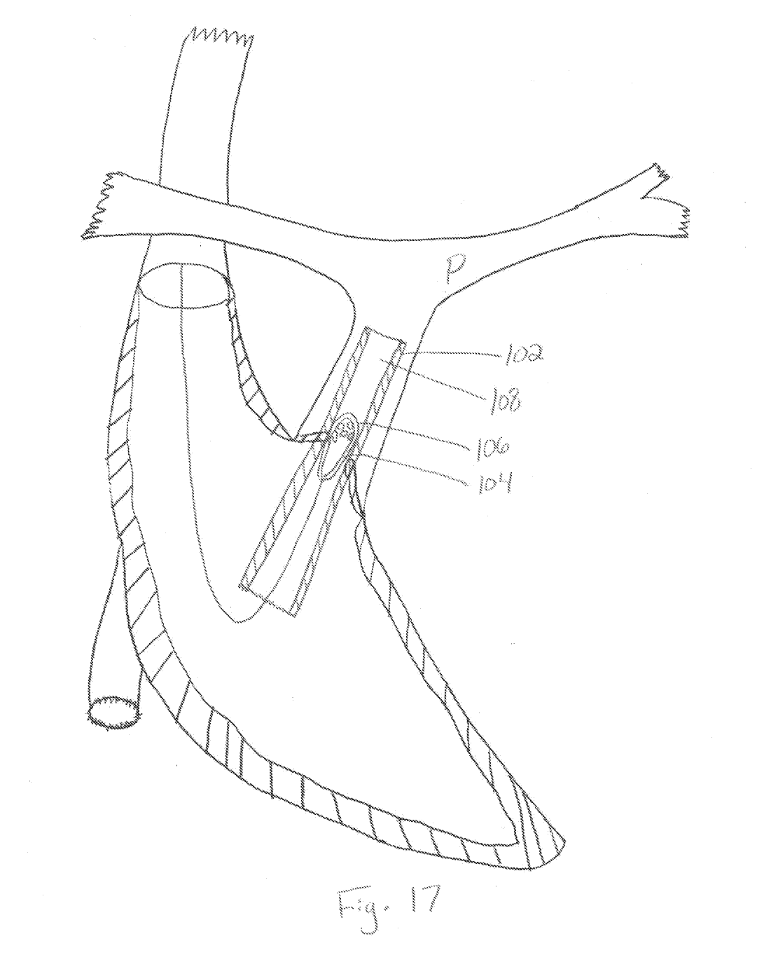

As shown in FIG. 14, the first drive shaft 828, when provided, may be rotated, in a similar manner to that previously described, and rotation of the first drive shaft 828 causes the first filter 104 to responsively rotate. Any provided second drive shaft 830 may also be rotated, in a similar manner to that previously described, with rotation of the second drive shaft 830 causing the second filter 106 to responsively rotate. The force of blood flow within the patient pulmonary artery P is utilized to restrict blood-carried emboli that are larger than the pores 526 of the moiré lattice structure 524 to a location on an upstream side of the moiré lattice structure 524 and thus catch blood-carried emboli on and/or in the moiré lattice structure 524. As shown in FIG. 15, an aspiration device 1554 may be inserted into the patient pulmonary artery P upstream of the embolic dual-filtration device 100. Emboli captured, alternatively referred to as restricted, within the moiré lattice structure 524 may be aspirated by the aspiration device 1554 away from the patient pulmonary artery P.

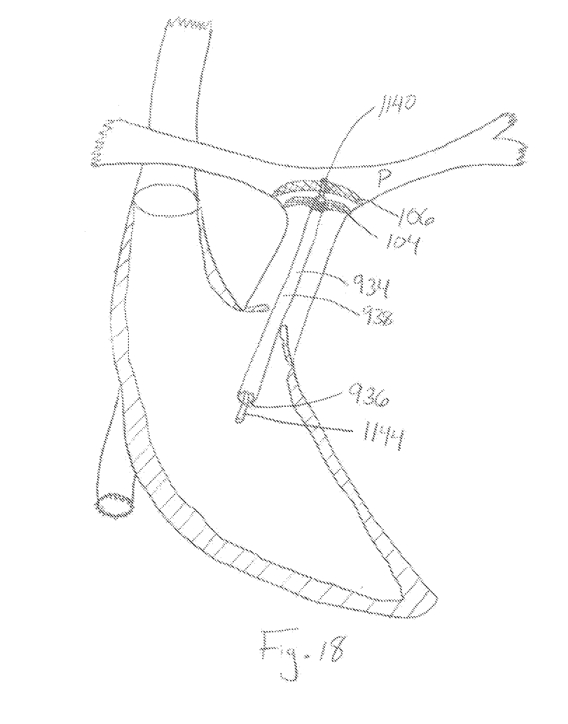

As shown in FIG. 16, the embolic dual-filtration device 100 may be collapsed into the collapsed condition, in any manner to that previously described. When the embolic dual-filtration device 100 is collapsed into the collapsed condition, the restricted emboli are maintained within the embolic dual-filtration device 100 as a result of the collapsed embolic dual-filtration device 100 at least partially surrounding the restricted emboli. As shown in FIG. 17, the first filter 104, the second filter 106, and the restricted emboli are removed from the patient pulmonary artery P through the catheter lumen 108.

As shown in FIGS. 16-17, the first and second filters 104, 106 collapsed over the restricted emboli may be longitudinally stretched so as to re-shape the volume of the first and second filters 104, 106 to be shaped to pass through the catheter lumen 108. The collapsed first and second filters 104, 106 with restricted emboli may be too large to pass through the catheter lumen 108. A force pulling the first and second filters 104, 106 from the patient pulmonary artery P and into the catheter lumen 108, in combination with the diameter of the catheter lumen 108 causes the first and second filters 104, 106 to longitudinally stretch in order to pass into and through the catheter lumen 108. In other words, as the collapsed first and second filters 104, 106 and restricted emboli, which are collectively too large to pass into the catheter lumen 108, are pulled from the patient pulmonary artery P and urged into the catheter lumen 108, the collapsed first and second filters 104, 106 are squeezed into the catheter lumen 108, causing the first and second filters 104, 106 and restricted emboli to longitudinally stretch. The emboli restricted to the upstream side of the moiré lattice structure 524 are removed from the patient pulmonary artery P when the first and second filters 104, 106 are removed from the patient pulmonary artery P.

As described above, the embolic dual-filtration device 100 may be provided with the first and second filter deployment tools 934, 1140. In use, the first filter 104 is collapsed into the collapsed condition on the first filter deployment outer wall 938 in a similar sequence to that previously described. With the first filter 104 in the collapsed condition, the first filter deployment tool 934 is inserted into the patient pulmonary artery P. As shown in FIG. 18, with the first filter 104 in the patient pulmonary artery P, the collapsed first filter 104 is expanded into the expanded condition in the patient pulmonary artery P in a similar sequence to that previously described. The second filter 106 is collapsed into the collapsed condition on the second filter deployment outer wall 1144 in a similar sequence to that previously described. With the second filter 106 in the collapsed condition, the second filter deployment tool 1140 is inserted through the first filter deployment tool inner lumen 936 and into the patient pulmonary artery P. With the second filter 106 in the patient pulmonary artery P, the collapsed second filter 106 is expanded into the expanded condition in the patient pulmonary artery P in a similar sequence to that previously described.

The embolic dual-filtration device 100 is maintained in the patient pulmonary artery P. With the embolic dual-filtration device 100 being maintained in the patient pulmonary artery P, the first and second filters 104, 106 are independently, selectively rotated, in a similar manner to that previously described, to form a moiré lattice structure 524 having varying sized pores 526 relative to the independent rotation of the first and second filters 104, 106. The force of blood flow is utilized within the patient pulmonary artery P to restrict blood-carried emboli that are larger than the pores 526 of the moiré lattice structure 524 to an upstream side of the moiré lattice structure 524. The embolic dual-filtration device 100 is collapsed into the collapsed condition in a similar sequence to that previously described. When the embolic dual-filtration device 100 is collapsed into the collapsed condition, the emboli are maintained within the embolic dual-filtration device 100 as a result of the collapsed embolic dual-filtration device 100 at least partially surrounding the emboli. The first filter 104, the second filter 106, and the restricted emboli are removed from the patient pulmonary artery P in a similar sequence to that previously described.

It is contemplated that the embolic dual-filtration device 100 may include a stretching tool (not shown) that is inserted into the patient pulmonary artery P to longitudinally stretch the collapsed embolic dual-filtration device 100 with restricted emboli to re-shape the volume of the embolic dual-filtration device 100 to a shape capable of passing through the catheter lumen 108.

It is contemplated that the first filter deployment tool 934 may have a first filter deployment tool side port (not shown). The first filter deployment tool side port may extend between the first filter deployment tool outer wall 938 and the first filter deployment tool inner lumen 936 to put the first filter deployment tool inner lumen 936 in fluid communication with the first filter deployment tool outer wall 938. The second filter deployment tool 1140 and attached second filter 106 may be configured to at least partially pass through the first filter deployment tool side port and into the first filter deployment tool inner lumen 936. In such case, with the second filter 106 in the collapsed condition, the second filter deployment tool 1140 may at least partially be inserted through the first filter deployment tool side port, through the first filter deployment tool inner lumen 936, and into the patient pulmonary artery P.

It is contemplated that the collapsed embolic dual-filtration device 100 may be configured to reduce the amount of captured emboli being extruded through the pores of the collapsed embolic dual-filtration device 100 or mitigate a “cheese grater” scraping effect. Instead of or in addition to reducing the amount of captured emboli being extruded, it is contemplated that the collapsed embolic dual-filtration device 100 may be configured to prevent captured emboli from extruding through the pores of the collapsed embolic dual-filtration device 100 or mitigate a “cheese grater” scraping effect. For example, a radial inward force may be applied by the collapsed embolic dual-filtration device 100 to hold the captured emboli on the moiré lattice structure 524, but without enough force to extrude the captured emboli through the pores 526.

It is contemplated that the size and/or a shape of at least one of the pores 216, 322 of the first and second filter meshes 214, 320, respectively, may be selectively adjusted in any desired manner, with or without an interaction by the user, such as by a mechanism that is triggered to automatically adjust the size and/or the shape of at least one of the pores 216, 322 of the first and second filter meshes 214, 320, respectively, and/or by the user manually adjusting the size and/or the shape of at least one of the pores 216, 322 of the first and second filter meshes 214, 320, respectively, directly, indirectly, or both.

The catheter 102, the first filter 104, the second filter 106, the first drive shaft 828, the second drive shaft 830, the first filter deployment tool 934, the second filter deployment tool 1140, the first anchoring member 1246, and/or the second anchoring member 1248 may each be at least partially formed from silicone, polyethylene, polypropylene, stainless steel, titanium, nitinol, any other shape memory alloy, any other biocompatible material, or any combination thereof.

The embolic dual-filtration device 100 assists the user in collecting emboli traveling through the patient pulmonary artery P. The embolic dual-filtration device 100 may assist the user in preventing embolization during a procedure such as, but not limited, to, a lead extraction, a percutaneous clot removal from the inferior vena cava, a percutaneous clot removal from the superior vena cava, or any suitable procedure.

Although the embolic dual-filtration device 100, to that previously described, as being used in a patient pulmonary artery P, it should be understood that the embolic dual-filtration device 100 may be used in any similar lumen to collect emboli or other undesirable matter traveling through that lumen.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

We claim:

1. An embolic dual-filtration device, comprising:
a first filter having pores; and
a second filter having pores, the second filter being positioned adjacent to the first filter, the first and second filters being capable of being selectively rotated with respect to one another;
wherein the first and second filter pores of the rotated first and second filters collectively form a moiré lattice structure having pores smaller than the pores of each of the separate first and second filters.

2. The embolic dual-filtration device of claim 1, wherein the first filter has a first filter support structure and a first filter mesh, the first filter support structure being capable of conforming to patient tissue geometry, the first filter mesh having pores and being attached to at least a portion of the first filter support structure.

3. The embolic dual-filtration device of claim 2, wherein the second filter has a second filter support structure and a second filter mesh, the second filter support structure being capable of conforming to the patient tissue geometry and the first filter, the second filter mesh having pores and being attached to at least a portion of the second filter support structure.

4. The embolic dual-filtration device of claim 1, including a catheter having a catheter lumen.

5. The embolic dual-filtration device of claim 4, wherein the first and second filters are each formed from a deformable material, the first and second filters each being capable of being selectively moved between collapsed and expanded conditions such that in the collapsed condition, the first and second filters each define a first size profile that is configured for passage through the catheter lumen to a desired location within a patient pulmonary artery, while in the expanded condition, the first and second filters each define a second size profile that is configured to engage a patient pulmonary artery tissue.

6. The embolic dual-filtration device of claim 5, wherein the deformable material is at least partially a shape memory alloy.

7. The embolic dual-filtration device of claim 6, including first and second drive shafts, the first drive shaft being attached to the first filter, the first drive shaft having a first drive shaft lumen, the second drive shaft being attached to the second filter, the second drive shaft being configured to fit within the first drive shaft lumen, wherein rotation of the first drive shaft causes the first filter to responsively rotate, and rotation of the second drive shaft causes the second filter to responsively rotate with respect to the first filter.

8. The embolic dual-filtration device of claim 1, including a first filter deployment tool, the first filter deployment tool having a first filter deployment tool inner lumen and a first filter deployment tool outer wall, the first filter deployment tool outer wall having the first filter attached thereon, the first filter being capable of being selectively moved between collapsed and expanded conditions around the first filter deployment tool outer wall.

9. The embolic dual-filtration device of claim 8, including a second filter deployment tool, the second filter deployment tool having a second filter deployment tool inner lumen and a second filter deployment tool outer wall, the second filter deployment tool outer wall having the second filter attached thereon, the second filter being capable of being selectively moved between collapsed and expanded conditions around the second filter deployment tool outer wall, the second filter deployment tool and attached second filter being configured to pass through the first filter deployment tool inner lumen when the second filter is in the collapsed position.

10. The embolic dual-filtration device of claim 1, wherein the average pore size of the first filter is larger than 250 microns, and the average pore size of the second filter is the same size as, or smaller than, the pores of the first filter.

11. The embolic dual-filtration device of claim 1, including a first anchoring member, the first anchoring member being attached to the first filter, the first anchoring member being capable of selectively anchoring the first filter to patient tissue.

12. The embolic dual-filtration device of claim 11, including a second anchoring member, the second anchoring member being attached to the second filter, the second anchoring member being capable of selectively anchoring the second filter to patient tissue.

13. The embolic dual-filtration device of claim 1, wherein each of the first and second filters is capable of selectively being attached to a guidewire.

14. The embolic dual-filtration device of claim 1, wherein the first and second filters are coaxially arranged relative to one another.

15. A system for collecting emboli in a pulmonary artery, comprising:
a first filter, the first filter having a first filter support structure and a first filter mesh, the first filter support structure being capable of conforming to patient pulmonary artery tissue geometry, the first filter mesh having pores and being attached to at least a portion of the first filter support structure;
a second filter, the second filter having a second filter support structure and a second filter mesh, the second filter support structure being capable of conforming to the patient pulmonary artery tissue geometry and the first filter, the second filter mesh having pores and being attached to at least a portion of the second filter support structure, the second filter being positioned longitudinally adjacent to the first filter, the first and second filters being coaxially arranged relative to one another, the first and second filters being capable of being rotated with respect to one another once positioned in the patient pulmonary artery; and
a catheter configured to access the patient pulmonary artery, the catheter having a catheter lumen, the catheter lumen being configured to allow the first and second filters to pass therethrough;
wherein the first and second filter meshes of the rotated first and second filters collectively form a moiré lattice structure having pores smaller than the pores of each of the separate first and second filters.

16. The embolic dual-filtration device of claim 15, wherein the first and second filters are each formed from a deformable material, the first and second filters each being capable of being selectively moved between collapsed and expanded conditions such that in the collapsed condition, the first and second filters each define a first size profile that is configured for passage through the catheter lumen to a desired location within a patient pulmonary artery, while in the expanded condition, the first and second filters each define a second size profile that is configured to engage a patient pulmonary artery tissue.

17. The embolic dual-filtration device of claim 16, wherein the deformable material is at least partially a shape memory alloy.

18. The embolic dual-filtration device of claim 15, including a first drive and second drive shaft, the first drive shaft being attached to the first filter, the first drive shaft having a first drive shaft lumen, the second drive shaft being attached to the second filter, the second drive shaft being configured to fit within the first drive shaft lumen, wherein rotation of the first drive shaft causes the first filter to responsively rotate, and rotation of the second drive shaft causes the second filter to responsively rotate with respect to the first filter.

19. The embolic dual-filtration device of claim 15, including a first filter deployment tool, the first filter deployment tool having a first filter deployment tool inner lumen and a first filter deployment tool outer wall, the first filter deployment tool outer wall having the first filter attached thereon, the first filter being capable of being selectively moved between collapsed and expanded conditions around the first filter deployment tool outer wall, the first filter deployment tool and attached first filter being configured to pass through the catheter lumen when the first filter is in the collapsed position.

20. The embolic dual-filtration device of claim 19, including a second filter deployment tool, the second filter deployment tool having a second filter deployment tool inner lumen and a second filter deployment tool outer wall, the second filter deployment tool outer wall having the second filter attached thereon, the second filter being capable of being selectively moved between collapsed and expanded conditions around the second filter deployment tool outer wall, the second filter deployment tool and attached second filter being configured to pass through the first filter deployment tool inner lumen and the catheter lumen when the second filter is in the collapsed position.

21. The embolic dual-filtration device of claim 15, wherein the average pore size of the first filter is larger than 250 microns, and the average pore size of the second filter is the same size as, or smaller than, the pores of the first filter.

22. The embolic dual-filtration device of claim 15, including a first anchoring member, the first anchoring member being attached to the first filter, the first anchoring member being capable of selectively anchoring the first filter to at least one of patient pulmonary artery tissue, patient right ventricle tissue, and patient right atrium tissue.

23. The embolic dual-filtration device of claim 22, including a second anchoring member, the second anchoring member being attached to the second filter, the second anchoring member being capable of selectively anchoring the second filter to at least one of patient pulmonary artery tissue, patient right ventricle tissue, and patient right atrium tissue.

24. The embolic dual-filtration device of claim 15, wherein each of the first and second filters is capable of selectively being attached to a guidewire.

25. A method for collecting emboli, the method comprising:
providing an embolic dual-filtration device having
a first filter having pores, and
a second filter having pores, the second filter being positioned adjacent to the first filter, the first and second filters being capable of being selectively rotated with respect to one another;
inserting the embolic dual-filtration device into a patient pulmonary artery;
maintaining the embolic dual-filtration device in the patient pulmonary artery;
with the embolic dual-filtration device being maintained in the patient pulmonary artery, selectively rotating the first and second filters independently to collectively form a moiré lattice structure having varying sized pores relative to the independent rotation of the first and second filters;
utilizing the force of blood flow within the patient pulmonary artery to restrict blood-carried emboli that are larger than the pores of the moiré lattice structure to a location on an upstream side of the moiré lattice structure; and
removing the first filter, the second filter, and the restricted emboli from the patient pulmonary artery;
wherein the emboli restricted to the upstream side of the moiré lattice structure are removed from the patient pulmonary artery when the first and second filters are removed from the patient pulmonary artery.

26. The method of claim 25, including:
providing a catheter, the catheter having a catheter lumen;
providing first and second filters formed from a deformable material, the first and second filters being capable of being selectively moved between collapsed and expanded conditions such that in the collapsed condition, the first and second filters each define a first size profile that is configured for passage through the catheter lumen to a desired location within a patient pulmonary artery, while in the expanded condition, the first and second filters each define a second size profile that is configured to engage a patient pulmonary artery tissue;
collapsing the embolic dual-filtration device into the collapsed condition;
inserting the catheter into the patient pulmonary artery;
with the embolic dual-filtration device in the collapsed condition, passing the embolic dual-filtration device through the catheter lumen and into the patient pulmonary artery;
with the embolic dual-filtration device in the patient pulmonary artery, expanding the embolic dual-filtration device into the expanded condition in the patient pulmonary artery;
maintaining the embolic dual-filtration device in the patient pulmonary artery;
with the embolic dual-filtration device being maintained in the patient pulmonary artery, selectively rotating the first and second filters independently to form a moiré lattice structure having varying sized pores relative to the independent rotation of the first and second filters;
utilizing the force of blood flow within the patient pulmonary artery to restrict blood-carried emboli that are larger than the pores of the moiré lattice structure to an upstream side of the moiré lattice structure; and
collapsing the embolic dual-filtration device into the collapsed condition, wherein the emboli are maintained within the embolic dual-filtration as a result of the collapsed embolic dual-filtration device at least partially surrounding the emboli.

27. The method of claim 25, including:
providing a first and a second drive shaft, the first drive shaft being attached to the first filter, the first drive shaft having a first drive shaft lumen, the second drive shaft being attached to the second filter, the second drive shaft being configured to fit within the first drive shaft lumen;
selectively rotating the first drive shaft, wherein rotation of the first drive shaft causes the first filter to responsively rotate; and
selectively rotating the second drive shaft, wherein rotation of the second drive shaft causes the second filter to responsively rotate with respect to the first filter.

28. The method of claim 25, including:
providing a first filter deployment tool, the first filter deployment tool having a first filter deployment tool inner lumen and a first filter deployment tool outer wall, the first filter deployment tool outer wall having the first filter attached thereon, the first filter being capable of being selectively moved between collapsed and expanded conditions around the first filter deployment tool outer wall;
providing a second filter deployment tool, the second filter deployment tool having a second filter deployment tool inner lumen and a second filter deployment tool outer wall, the second filter deployment tool outer wall having the second filter attached thereon, the second filter being capable of being selectively moved between collapsed and expanded conditions around the second filter deployment tool outer wall, the second filter deployment tool outer wall and attached second filter being configured to pass through the first filter deployment tool inner lumen when the second filter is in the collapsed condition;
collapsing the first filter into the collapsed condition on the first filter deployment outer wall;
with the first filter in the collapsed condition, inserting the first filter deployment tool into the patient pulmonary artery;
with the first filter in the patient pulmonary artery, expanding the collapsed first filter into the expanded condition in the patient pulmonary artery;
collapsing the second filter into the collapsed condition on the second filter deployment outer wall;
with the second filter in the collapsed condition, inserting the second filter deployment tool through the first filter deployment tool inner lumen and into the patient pulmonary artery;
with the second filter in the patient pulmonary artery, expanding the collapsed second filter into the expanded condition in the patient pulmonary artery;
maintaining the dual-filter device in the patient pulmonary artery;
with the embolic dual-filtration device being maintained in the patient pulmonary artery, selectively rotating the first and second filters independently to form a moiré lattice structure having varying sized pores relative to the independent rotation of the first and second filters;
utilizing the force of blood flow within the patient pulmonary artery to restrict blood-carried emboli that are larger than the pores of the moiré lattice structure to an upstream side of the moiré lattice structure; and
collapsing the embolic dual-filtration device into the collapsed condition, wherein the emboli are maintained within the embolic dual-filtration as a result of the collapsed embolic dual-filtration device at least partially surrounding the emboli.

29. The method of claim 25, including:
providing a first anchoring member, the first anchoring member being attached to the first filter, the first anchoring member being capable of selectively anchoring the first filter to at least one of patient pulmonary artery tissue, patient right ventricle tissue, and patient right atrium tissue;
inserting the first filter into a patient pulmonary artery; and
maintaining the first filter in the patient pulmonary artery by selectively anchoring the first filter to patient tissue;
wherein when the first anchoring member is anchored to patient tissue, the first filter is restricted from egressing from the patient pulmonary artery.

30. The method of claim 29, including:
providing a second anchoring member, the second anchoring member being attached to the second filter, the second anchoring member being capable of selectively anchoring the second filter to at least one of patient pulmonary artery tissue, patient right ventricle tissue, and patient right atrium tissue;
inserting the second filter into a patient pulmonary artery; and
maintaining the second filter in the patient pulmonary artery by anchoring the second filter to patient tissue;
wherein when the second anchoring member is anchored to patient tissue, the second filter is restricted from egressing from the patient pulmonary artery.
